(19) United States
(12) Patent Application Publication
(10) Pub. No.: US 2009/0038615 A1
(43) Pub. Date: Feb. 12, 2009

(54) BREATHING APPARATUS

(75) Inventor: Austen Bradley, Daresbury (GB)

Correspondence Address: JAMES D. STEVENS;REISING, ETHINGTON, BARNES, KISSELLE, P.C., P.O. BOX 4390, TROY, MI, 48099, US

(73) Assignee: MEDITREN LIMITED, Daresbury, Cheshire (UK)

(21) Appl. No.: 12/280,436

(22) PCT Filed: Feb. 23, 2007
(86) PCT No.: PCT/GB07/00657

§ 371 Date: Aug. 22, 2008

(30) Foreign Application Priority Data
Feb. 23, 2006 (GB) 0603599.2

(51) Int. Cl. A61M 16/00

(52) U.S. Cl. 128/204.17

(57) ABSTRACT

A breathing apparatus and a method of reducing condensation within a breathing apparatus. The apparatus (100) includes a respiratory device (112a, 112b) arranged to process respiratory gases, a conduit (116) for input of respiratory gases to the respiratory device, and a temperature-altering device (120) arranged to alter the temperature of the respiratory gases for reduction of condensation within the respiratory gas device.

The present invention relates to a breathing apparatus, and in particular to a breathing apparatus including an arrangement for reducing condensation within the apparatus.

Breathing apparatus are apparatus into which a subject (human or animal) can breath. Breathing apparatus can be utilised to facilitate breathing e.g. artificial ventilators. Equally, breathing apparatus can be utilised to measure one or more respiratory parameters. For example, the volume and flow rate of inhaled or exhaled breath may be monitored using a respiratory spirometer. Indirect calorimeters can be used to measure the resting metabolic rate of a person by measuring the expired or exhaled airflow from a subject, including measuring the carbon dioxide and oxygen concentration therein.

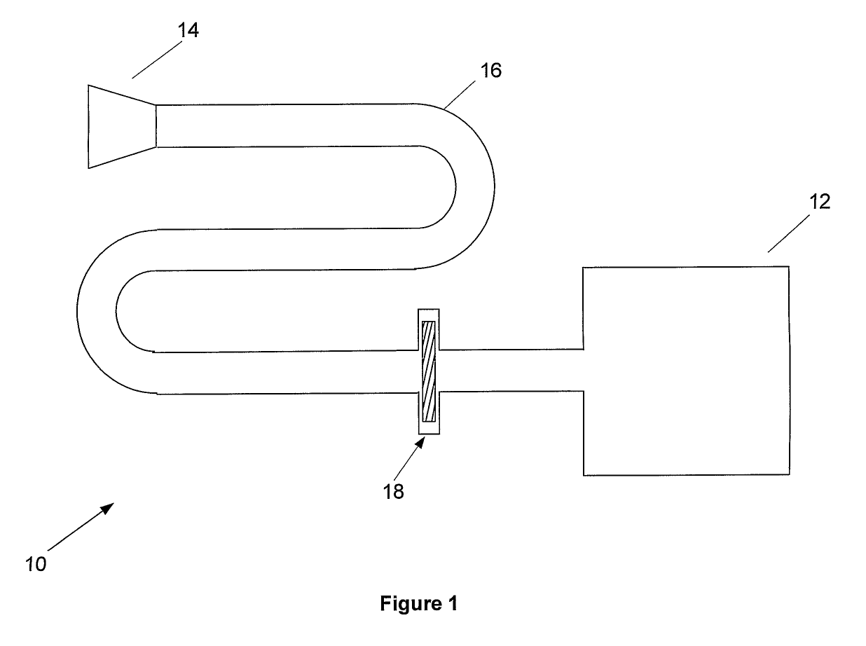

FIG. 1 shows a schematic diagram of a typical breathing apparatus 10. The apparatus 10 includes a main body 12 in which respired gases are processed e.g. measured. The apparatus further includes a mouthpiece 14 coupled to the main body 12 via a flexible tube 16. Respiratory gases enter the main body 12 of the breathing apparatus via a subject breathing into the mouthpiece 14, and the respiratory gases travelling along tube 16. Exhaled breath has a high humidity, due to absorbing moisture from within the lungs. To inhibit/reduce condensation forming within the main body 12, a moisture trap or filter 18 is located along the flow path of the respiratory gases.

The moisture trap or filter 18 can take the form of a chemical trap or a physical filter, and is arranged to remove moisture from the respiratory gases. However, utilising such a chemical or physical trap/filter is undesirable. The trap/filter provides a resistance to the flow of gases, thus creating a backpressure against the exhaled breath. Such a backpressure is undesirable, as it can impede/alter the breathing rhythm or exhalations of a subject.

One known method of overcoming the backpressure effect of the moisture trap/filter is to provide a pump, to pull the respiratory gases through the filter. Typically, the pumping system will utilise room air to dilute the respiratory gases, thus altering the concentration of gases within the exhaled air, and potentially reducing the accuracy of any subsequent replenishment of those gases. However, providing such a pump to a breathing apparatus increases the complexity of the apparatus, leading to an increase in manufacturing and maintenance costs. Further, the operation of the pump may still alter the breathing rhythm of a subject.

It is an aim of the embodiments of the present invention to address one or more problems of the prior art, whether referred to herein or otherwise.

In a first aspect, the present invention provides a breathing apparatus comprising a respiratory device arranged to process respiratory gases; a conduit for input of respiratory gases to said respiratory device; and a temperature-altering device arranged to alter the temperature of said respiratory gases for reduction of condensation within said respiratory gas device.

Providing such a temperature-altering device within a breathing apparatus allows the temperature of the respiratory gases to be altered so as to prevent or reduce condensation. The gases can be heated by the temperature-altering device, so as to reduce the likelihood of, or decrease the extent to which, the respiratory gases condense within the respiratory gas device. Equally, the gases could be cooled, so as to condense water vapour from the respiratory gases at a desired position within the breathing apparatus, so as to limit the condensation that occurs within the respiratory gas device. If desired, the temperature altering device could be arranged to first cool the respiratory gases (so as to remove some water vapour), and then to subsequently warm the respiratory gases to decrease the likelihood, or extent of, further condensation.

Said temperature-altering device may comprise at least one heater for warming the respiratory gases.

Said heater may be arranged to warm said respiratory gases to a temperature of at least 35° C.

The breathing apparatus may further comprise a flow meter for sensing the flow rate of the respiratory gases, wherein said heater is arranged to heat respiratory gases passing through said flow meter.

Said temperature-altering device may comprise a cooling device arranged to cool said respiratory gases to condense water vapour from the gases.

Said cooling device may be arranged to cool said respiratory gases to a temperature of 25° C. or less.

Said cooling device may comprise at least one cooling surface positioned adjacent a portion of said conduit.

Said at least one cooling surface may comprise a peltier cooler.

The breathing apparatus may comprise two of said cooling surfaces positioned in mutual opposition, adjacent the same portion of said conduit.

Said portion of the conduit may comprise a removable cartridge for removal of condensed water vapour.

Said removable cartridge may be positioned inline within the conduit such that all of the respiratory gases entering the respiratory gas device pass through the cartridge.

Said conduit may further comprise a tube, and said cartridge may comprise a first connector for connection to said tube and a second connector for connection to the respiratory gas device.

Said cartridge may have a cross-sectional area substantially equal to the cross-sectional area of said tube.

Said respiratory gas device may comprise at least one of: a gas sensor for sensing the presence of a gas within the respiratory gases; a pressure sensor for sensing the pressure of the respiratory gases; and a flow meter for sensing the flow rate of the respiratory gases.

In a second aspect, the invention provides a cartridge for use in a breathing apparatus.

Said cartridge may include gas directing means for direction of gas towards a surface of the cartridge.

Said cartridge may comprise two parallel, substantially planar outer surfaces, each surface being positioned adjacent a respective cooling surface of the cooling device when in use.

In a third aspect the present invention provides a method of reducing condensation within a breathing apparatus arranged to process respiratory gases, and may comprise controlling a device to alter the temperature of said respiratory gases.

Said temperature may be increased so as to reduce the likelihood of water vapour condensing from said respiratory gases.

Said temperature may be reduced, so as to condense water vapour from the respiratory gases at a desired position within the breathing apparatus.

An embodiment of the present invention will now be described, by way of example only, with reference to the accompanying drawings, in which:

FIG. 1 is a schematic diagram of a known breathing apparatus;

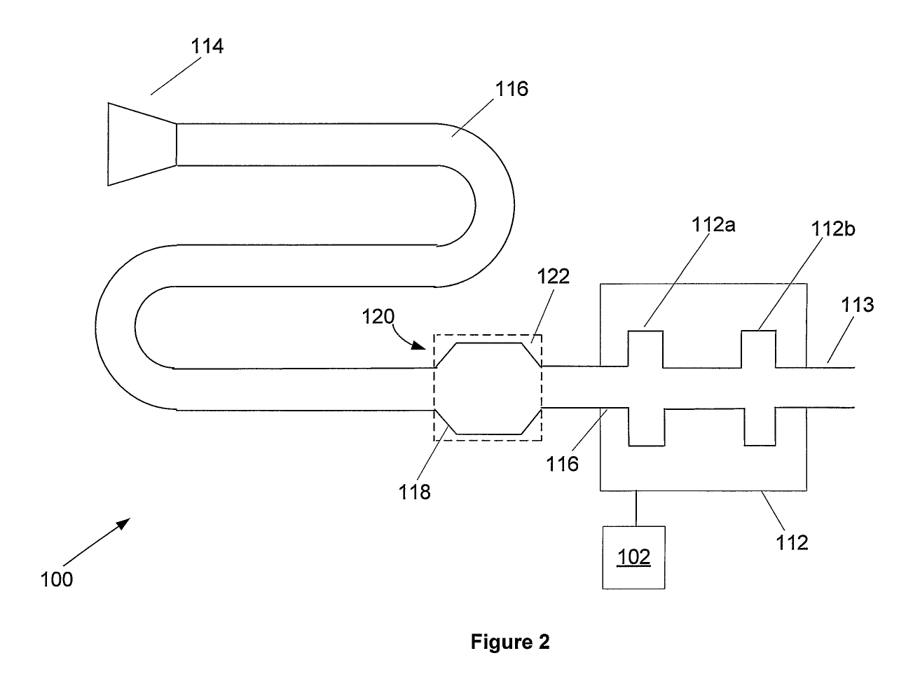

FIG. 2 is a schematic diagram of a breathing apparatus in accordance with an embodiment of the present invention;

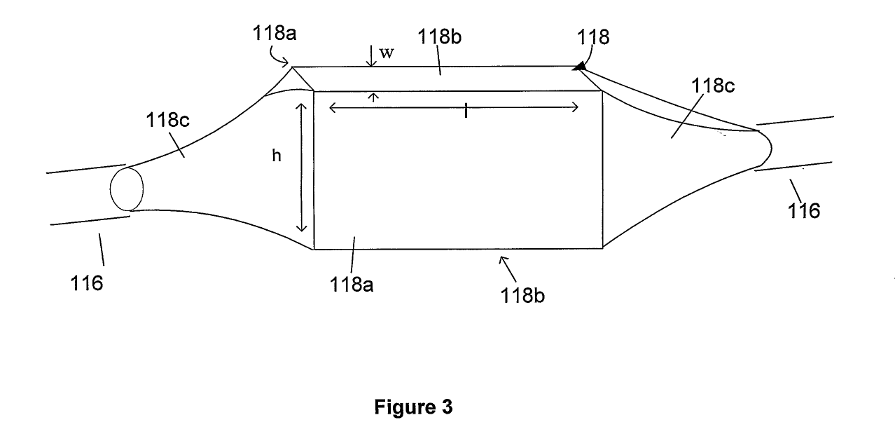

FIG. 3 is a perspective view of the cooling cartridge shown in FIG. 2 in accordance with an embodiment of the present invention;

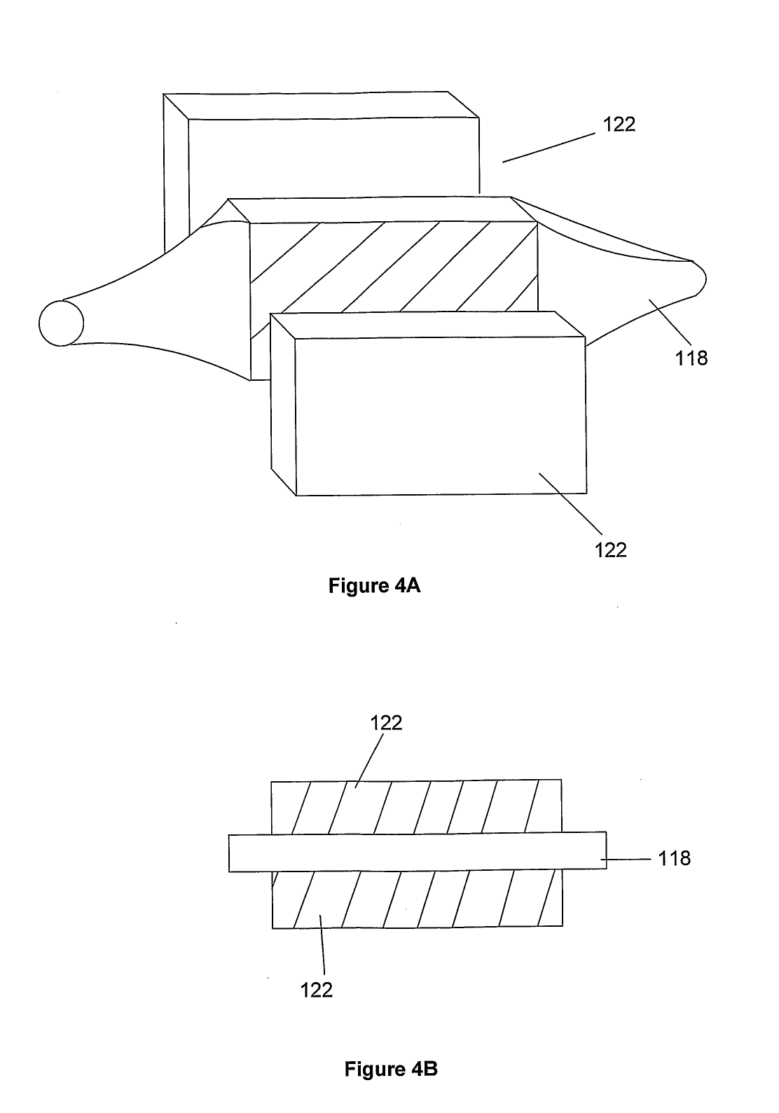

FIGS. 4A and 4B are respectively a perspective view and a plan cross-sectional view of the cooling cartridge of FIG. 3 located between two cooling surfaces; and

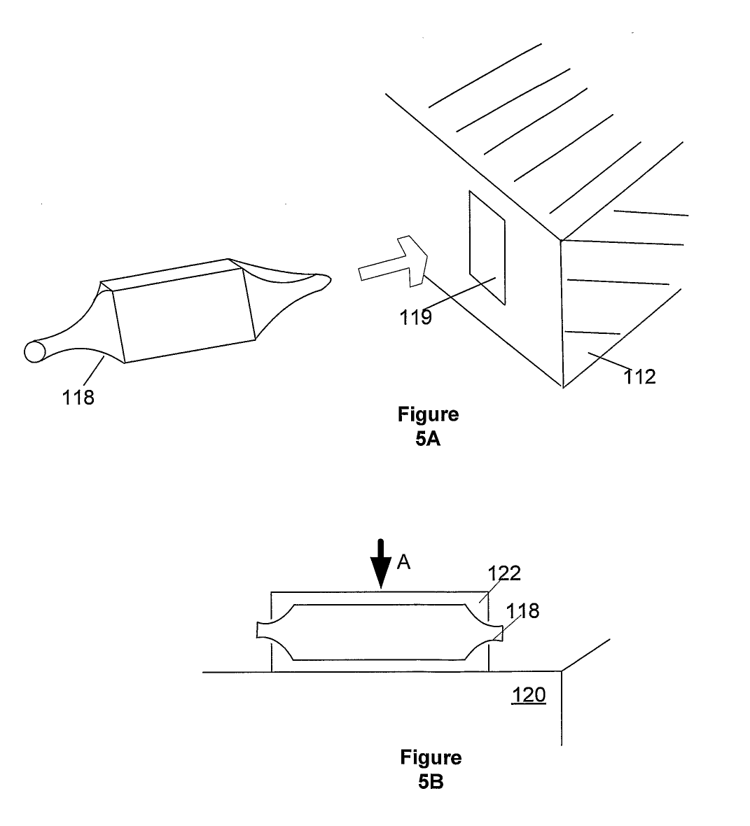

FIGS. 5A and 5B illustrate alternative cartridge insertion arrangements in accordance with embodiments of the present invention.

FIG. 2 show a breathing apparatus 100 including a temperature altering device 118, 120 in accordance with an embodiment of the present invention.

The apparatus 100 includes a body 112 housing respiratory gas devices 112a, 112b to process respiratory gases. In this particular embodiment, the breathing apparatus acts as an indirect calorimeter. The respiratory gas devices consist of a flow meter 112a and oxygen and carbon dioxide gas sensors 112b.

The respiratory gas devices 112a, 112b are connected to a mouthpiece 114 via a conduit 116, 118. Both devices 112a, 112b are connected in series between the mouthpiece 114 and an exhaust tube 113 which vents to atmosphere. In this particular embodiment, the breathing apparatus 100 acts as a free breathing device, allowing a subject to both inhale and exhale through the mouthpiece 114. The mouthpiece 14 includes two one-way valves, typically located within a T piece. The first one-way valve opens to the atmosphere, and the second one-way valve to conduit 116. As the subject inhales, the first one-way valve opens, and allows the subject to draw air in from the atmosphere, whilst the second valve stays shut. As the subject exhales, the first valve to the atmosphere shuts, and the second valve to conduit 116 opens. Thus, on the subject breathing out, the exhaled breath travels through the second valve of the mouthpiece 114, along conduit 116, 118, through devices 112a, 112b, and hence to atmosphere via exhaust tube 113. As this is a free breathing device, no mechanical assistance is provided to the breathing action of a subject by the breathing apparatus 100.

Flow meter 112a is arranged to measure the flow rate of respiratory gases. In this embodiment, the flow meter 112a is a thermal mass flow meter, a device known in the art. Preferably, the conduit 116, 118 is arranged to provide laminar flow of exhaled respiratory gases to the flow meter 112a, so as to increase the accuracy of the measurement performed by the flow meter. For example, gas directing means (e.g. fins, grooves or vanes) could be provided in a portion of the conduit 118 so as to direct exhaled gases towards laminar flow upon exiting that portion of the conduit 118.

Sensors 112b are arranged to measure the concentration of oxygen and carbon dioxide in the respiratory gas. A display 102 displays the results of the measurement by the flow meter 112a and the sensors 112b.

Typically, the average temperature of a human body is approximately 37° C. i.e. at a higher temperature than a typical room. For example, rooms within many buildings are maintained at a temperature of around 18-20° C. Exhaled respiratory gases typically leave the body of a subject at a temperature close to body temperature, due to the gases having been warmed whilst within the lungs of the subject. Further, gases are typically exhaled at a high relative humidity i.e. approaching 100% humidity. As exhaled respiratory gases are cooled by the environment towards room temperature, for instance as the gases pass through the breathing apparatus, water vapour within the exhaled respiratory gases condenses out. Condensation within the breathing apparatus, particularly within the respiratory gas devices 112a, 112b enclosed within the body 112, is undesirable. Droplets of condensation can lead to degradation of the performance of the respiratory gas devices e.g. inaccurate measurements of the properties of the gases. Further, condensed moisture can lead to spreading of diseases and infections between different subjects, when different subjects utilise the breathing apparatus.

To address this problem, the present invention includes a temperature altering device 120 positioned adjacent a portion of the conduit 118. In this particular embodiment, the temperature altering device 120 comprises two parallel, planar surfaces 122 (shown in dotted outline in FIG. 2), positioned adjacent a portion 118 of the conduit. The temperature of surfaces 122 is controlled so as to control the temperature of gases within the breathing apparatus.

The temperature altering device can be arranged to heat the exhaled respiratory gases within the conduit portion 118 by several degrees centigrade (e.g. approximately 5° C. or 10° C.), so as to decrease the likelihood of water vapour condensing from the exhaled respiratory gases after they exit the portion 118 towards respiratory gas devices 112a, 112b.

In this particular embodiment, the temperature altering device 120 is arranged to cool the exhaled respiratory gases, so as to condense some water vapour from the gases prior to the gases entering respiratory gas devices 112a, 112b. For example, the temperature altering device may be arranged to cool the expired respiratory gases by approximately 5° C.-10° C.

It will be appreciated that any number of cooling devices could be utilised to act as the temperature altering device 120. However, preferably each cooling surface 122 is provided by one or more peltier devices i.e. devices that operate via the peltier effect.

Typically, the remainder of the conduit 116 takes the form of a plastic tube. The plastic tube is typically of circular cross-section, with a diameter of approximately 22 mm. Typically, the tubing 116 between the mouthpiece 114 and the cartridge 118 will be formed as removable, disposable tubing, as condensation may form within that portion of the conduit.

Portion 118 is preferably formed as a removable cartridge, for removable attachment to the rest of the breathing apparatus. The cartridge 118 comprises a first connector 118c arranged for connection to the conduit 116 (coupled to mouthpiece or mask 114) utilised to input exhaled respiratory gases, and a second connector 116 for coupling to the respiratory gas devices 112a, 112b. In the particular embodiment illustrated in FIGS. 2 and 3, a further section of tubing 116 is utilised to couple the cartridge 118 to the flow meter 112a. The connectors are simply push fit connections, suitably sized to receive the tubing 116.

Preferably, the cartridge 118 has an internal cross-sectional area substantially equal to the internal cross-sectional area of the remainder of the conduit 116. The cross-sectional area of the cartridge 118 is the area of the cartridge as measured in a plane substantially perpendicular to the bulk direction of flow of the respiratory gases. To increase the rate of heat transfer (e.g. heating or cooling) between the surfaces of the temperature altering device 122 and the cartridge 118, the central portion of the cartridge (118a, 118b) is cuboidal. Two first parallel, relatively large planar surfaces 118a are arranged, in use, to lie adjacent a respective surface 122 of the temperature altering device 120. Two other parallel surfaces 118b extend in respective planes substantially perpendicular to surfaces 118a, connecting surfaces 118a. Surfaces 118b are of width w and of length 1. Typically, width w of surface 118b is smaller than height h of surface 118a, so as to maximise the heat exchange process between the gas and the surfaces 122 of the temperature altering device. Typically, 1 will be approximately 60 mm, h approximately 30 mm, and w approximately 13 mm.

First and second connectors 118c are shaped, at the distal ends of the cartridge 118, so as to couple to tubes 116 and transition gradually in configuration (i.e. taper) to form the cuboidal central body portion defined by surfaces 118a, 118b. The removable cartridge can be formed of any suitable material, including plastic. Preferably, the cartridge is used as a disposable cartridge, and disposed of after the breathing apparatus has been used by each subject. Equally, the cartridge can be disposed of after each treatment and/or measurement relating to a particular subject.

Typically, to enhance heat transfer, gas deflecting means (e.g. vanes, grooves or fins) will be formed within the portion of the cartridge 118 through which exhaled gases enter the cartridge (e.g. connector 118c), so as to promote movement of the gases to adjacent the large surfaces 118a. Further gas deflecting means may also be provided at the exit of the cartridge 118, so as to promote laminar flow within the exhaled respiratory gases exiting the cartridge 118 towards the gas flow meter.

As indicated in FIGS. 5A and 5B, the cartridge 118 may be inserted into the breathing apparatus in a number of different configurations. For example, FIG. 5A illustrates how the cartridge 118 can be inserted into the body 112 of the breathing apparatus via a suitable slot 119. In that embodiment, the temperature-altering device is located within the body 112. Alternatively, in the configuration shown in FIG. 5B, the cartridge 118 may (in use) be located outside of the body 112, and mounted between the two surfaces 122, with the two surfaces 122 being affixed to a surface provided by the temperature altering device 120.

Although the temperature-altering device has been illustrated as being located adjacent the body of the breathing apparatus (i.e. adjacent the flow meter in the preferred embodiment), it will be appreciated that the temperature-altering device could be located elsewhere within the breathing apparatus. For example, the temperature-altering device could be located so as to warm the respiratory gases as the gases pass through the flow meter. Equally, the temperature-altering device could be arranged to heat gases immediately prior to the gases passing through the flow meter. Alternatively, a cooling device could be arranged adjacent the mouthpiece 114, so as to cool the respiratory gases immediately after leaving the mouthpiece, prior to entering the conduit to the breathing apparatus.

In the above embodiment, the temperature-altering device has been indicated as comprising two substantially planar, parallel surfaces, with a cartridge (which functions as a portion of the conduit) being disposed between the two surfaces. However, it will be appreciated that the temperature-altering device could be implemented in a number of different configurations. Equally, the portion of the conduit cooled (or heated) by the temperature-altering device could be implemented in a number of different configurations. For example, the temperature-altering device could take the form of a temperature-altering tube or jacket extending around the conduit (e.g. extending around the tube in the above embodiment). Alternatively, the temperature-altering device could take the form of a chamber enclosing the relevant portion of the conduit, thus acting like a miniature oven or refrigerator. The relevant portion of the conduit affected by the temperature-altering device could be a cartridge as described above, or could take the form of a chamber that forms an integral part of the conduit, or could simply be a portion of the conduit (e.g. a predetermined section or length of the tube).

Providing a breathing apparatus including such a temperature-altering device as described herein, provides a reduction in the condensation that forms within the breathing apparatus, leading to an improvement in the performance of the breathing apparatus, as well as a decrease in the risk of infection being transmitted between different subjects.

1. A breathing apparatus comprising: a respiratory device arranged to process respiratory gases; a conduit for input of respiratory gases to said respiratory device; and a temperature-altering device arranged to alter the temperature of said respiratory gases for reduction of condensation within said respiratory gas device.

2. A breathing apparatus as claimed in claim 1, wherein said temperature-altering device comprises at least one heater for warming the respiratory gases.

3. A breathing apparatus as claimed in claim 2, wherein said heater is arranged to warm said respiratory gases to a temperature of at least 35° C.

4. A breathing apparatus as claimed in claim 1, and further comprising a flow meter for sensing the flow rate of the respiratory gases, wherein said heater is arranged to heat respiratory gases passing through said flow meter.

5. A breathing apparatus as claimed in claim 1, wherein said temperature-altering device comprising a cooling device arranged to cool said respiratory gases to condense water vapour from the gases.

6. A breathing apparatus as claimed in claim 5, wherein said cooling device is arranged to cool said respiratory gases to a temperature of 25° C. or less.

7. A breathing apparatus as claimed in claim 5, wherein said cooling device comprises at least one cooling surface positioned adjacent a portion of said conduit.

8. A breathing apparatus as claimed in claim 7, wherein said at least one cooling surface comprises a peltier cooler.

9. A breathing apparatus as claimed in claim 7, comprising two of said cooling surfaces positioned in mutual opposition, adjacent the same portion of said conduit.

10. A breathing apparatus as claimed in claim 7, wherein said portion of the conduit comprises a removable cartridge for removal of condensed water vapour.

11. A breathing apparatus as claimed in claim 10, wherein said removable cartridge is positioned inline within the conduit such that all of the respiratory gases entering the respiratory gas device pass through the cartridge.

12. A breathing apparatus as claimed in claim 10, wherein said conduit further comprises a tube, and said cartridge comprises a first connector for connection to said tube and a second connector for connection to the respiratory gas device.

13. A breathing apparatus as claimed in claim 12, wherein said cartridge has a cross-sectional area substantially equal to the cross-sectional area of said tube.

14. A breathing apparatus as claimed in claim 1, wherein said respiratory gas device comprises at least one of: a gas sensor for sensing the presence of a gas within the respiratory gases; a pressure sensor for sensing the pressure of the respiratory gases; and a flow meter for sensing the flow rate of the respiratory gases.

15. A cartridge for use in a breathing apparatus as claimed in claim 10.

16. A cartridge as claimed in claim 15, wherein said cartridge includes gas directing means for direction of gas towards a surface of the cartridge.

17. A cartridge as claimed in claim 15, wherein said cartridge comprises two parallel, substantially planar outer surfaces, each surface being positioned adjacent a respective cooling surface of the cooling device when in use.

18. A method of reducing condensation within a breathing apparatus arranged to process respiratory gases, comprising controlling a device to alter the temperature of said respiratory gases.

19. A method as claimed in claim 18, wherein said temperature is increased so as to reduce the likelihood of water vapor condensing from said respiratory gases.

20. A method as claimed in claim 18, wherein said temperature is reduced, so as to condense water vapour from the respiratory gases at a desired position within the breathing apparatus.
